(19) United States
(12) Patent Application Publication
(10) Pub. No.: US 2012/0052102 A1
(43) Pub. Date: Mar. 1, 2012

(54) METHOD FOR PRODUCING COSMETICS

(75) Inventor: Rudolf Erwin Berghoff, Unterschleissheim (DE)

(73) Assignee: LINDE AKTIENGESELLSCHAFT, München (DE)

(21) Appl. No.: 13/254,482

(22) PCT Filed: Mar. 4, 2010
(86) PCT No.: PCT/EP2010/001361

§ 371 Date: Nov. 10, 2011

(30) Foreign Application Priority Data
Mar. 6, 2009 (DE) 10 2009 012 068.8
Mar. 6, 2009 (DE) 10 2009 012 069.6
Mar. 27, 2009 (EP) 09004501.4

(51) Int. Cl. A61K 8/02; A61K 8/92; A61Q 19/00; A61Q 5/02; A61Q 17/04; A61K 8/06; A61Q 5/00

(52) U.S. Cl. 424/401; 514/63; 424/59; 424/70.1

(57) ABSTRACT

A method is provided for producing a cosmetic or pharmaceutical product, wherein a plurality of components are processed to form the product and at least one component in a liquid state is atomized, shock-cooled and converted into a granulate. The granulate is processed with further components to form the product.

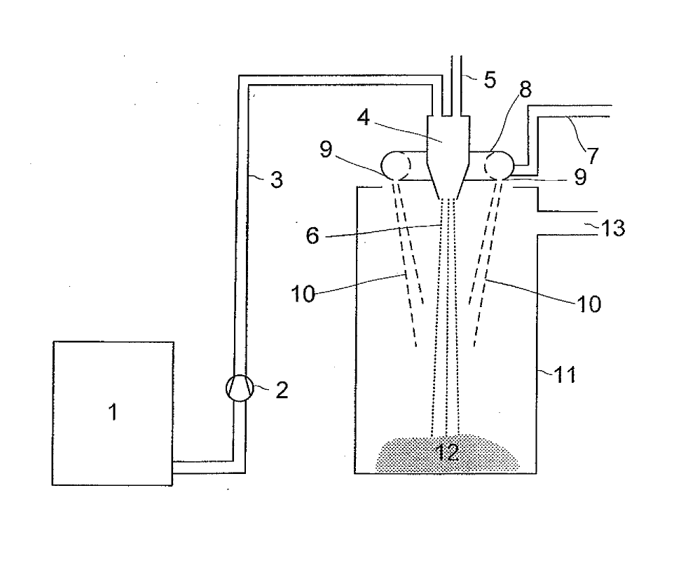

The invention relates to a method for producing a cosmetic or pharmaceutical product, wherein a plurality of components are processed to form the product, wherein at least one component in the liquid state is atomized, cooled and converted into a granulate, and the granulate is processed with the further components to form the product.

When producing creams, pastes and similar cosmetic products, fat-containing components are mixed and processed with further components necessary for the product, in particular additives and active ingredients, and are often also mixed and processed with aqueous components. The aqueous and fatty components do not, or only hardly, dissolve in one another and thus form a dispersion once mixed. Suitable emulsifiers and stabilizers are used to prevent a rapid segregation of the dispersion.

A melt crystallization method is known from EP 1 923 188 A1 in which a melt is added at the head of a cooling pipe and is separated into uniform drops. The drops fall downwards and are contacted with a cold gas flow, wherein the drops crystallize to form a granulate having a diameter of up to 2 mm. This melt crystallization method is also referred to as prilling.

In cosmetic products the individual product components are to be distributed as finely as possible. Granulate produced in accordance with the above-described method is unsuitable for cosmetic products, or is only suitable to a limited extent, owing to the particle size. It is also more advantageous for the production of cosmetics to provide the individual product components as an amorphous structure instead of a crystalline granulate.

The object of the present invention is to present a method for producing cosmetic or pharmaceutical products which allows a finer dispersion of the individual product components and greater stability of the product.

In particular, the atomized component is to be converted into a very fine granulate which has an amorphous structure where possible.

This object is achieved by a method for producing a cosmetic or pharmaceutical product, wherein a plurality of components are processed to form the product, wherein at least one component in the liquid state is atomized, cooled and converted into a granulate, and wherein the granulate is processed with the further components to form the product, wherein the at least one component in the liquid state is shock-cooled by direct heat exchange with a cryogenic liquid gas, preferably with liquid nitrogen.

The term “cosmetic product” is to be understood to mean all types of nourishing, cleansing and decorative cosmetic products. For example, it includes means for cleaning skin, hair or teeth, such as cleansing lotions, bath oils, skin creams, body lotions, lip care products, shampoos or toothpastes. However, it also includes means for beautifying skin or hair, such as tinted creams, eye care products, lipsticks or hair gels. In particular, the term “cosmetic product” includes fat-containing products, such as creams, ointments, pastes, emulsions and suspensions.

The term “granulate” in particular includes a particulate, pulverulent or granular material.

In accordance with the invention at least one of the components used for the cosmetic or pharmaceutical product is first brought into the liquid state of matter. For this purpose, provided it is not already present in the liquid state of matter, the component is either melted or dissolved in a solvent, such as water or oil, or in other formulation ingredients. The liquid thus obtained is then is then atomized, wherein it is separated into very fine droplets. These very fine liquid droplets are cooled suddenly by direct heat exchange with a cryogenic liquid gas, i.e. by being contacted with a cryogenic liquid, so that the droplets are converted into the solid state of matter without agglomeration and form a granular or pulverulent phase.

Owing to the direct contact with a cryogenic liquid, the fine liquid droplets are cooled approximately 100 to 1000 times quicker than by heat exchange with a cold gas. Owing to the sudden cooling, the droplets preferably solidify in the desired amorphous structure, and not in a crystalline structure.

The at least one component is preferably atomized in such a way that a fine spray is produced. In addition, the spray and the cryogenic liquid gas are strongly swirled with one another. In the prilling method described above, crystalline pearls which are as equal in size as possible are to be produced. For this purpose it is necessary for the drops to fall downwards in the cooling pipe with as few interruptions as possible. In the known prilling method, although cooled by the fed cold gas, the drops should not be swirled or broken, however, since otherwise pearls of different sizes will be formed.

By contrast, in accordance with the invention the component to be granulated is atomized in such a way that it interacts as strongly as possible with the cryogenic liquid so as to solidify as quickly as possible. This is achieved both by the use of a cryogenic liquid gas, that is to say a cryogenic liquid, and by the strong swirling of the droplets with the cryogenic liquid.

Before the component is atomized, it must be liquefied if it is not already present in the liquid state. For example, this may be achieved by melting or by dissolution in a solvent. In the case of melting, the component is preferably heated only to the minimum temperature required for the subsequent spraying process in order to save energy. If the component is instead dissolved in a solvent, only the minimum amount of solvent needed is advantageously used.

A particulate, pourable material which can be processed further in a quick and simple manner is obtained by the method according to the invention. The granulate is mixed with the further components and is optionally subjected to further processing steps, such as heating. When using the granulate according to the invention, the individual components are distributed in the cosmetic or pharmaceutical product in a much more finely dispersed manner than with conventional production methods. The homogeneity and stability of the products is considerably improved.

In a preferred embodiment the component in the liquid state is subjected to increased pressure, is fed to a spraying nozzle and is atomized. It is also possible to feed the liquid component to a nozzle by means of a propellant, via which nozzle the component is emitted and finely distributed. In this case the liquid component can be combined with the propellant in a basically unpressurized manner. Gaseous nitrogen is preferably used as a propellant. Other atomization and spraying techniques may also be used. The droplet size, and thus the particulate size of the granulate produced during the subsequent cooling is advantageously set by the type of spraying technique, the selection of the spraying nozzle and by the spraying parameters, such as the pressure and speed of the liquid component directly before the atomizing process.

The liquefied component may be atomized using single- or two component nozzles. When using single-component nozzles pressures between 20 and 70 bar are necessary depending on the desired drop/particle size, and even up to 300 bar with very small particle sizes. In two-component nozzles the liquid can be conveyed at low pressures between approximately 2 and 8 bar. The pressure of the gas flow, which is what causes the atomization, is normally approximately 1 to 2 bar higher.

The at least one component is advantageously cooled by direct heat exchange with liquid nitrogen. Owing to the cooling with liquid nitrogen, the droplets solidify very rapidly to form a fine granulate. The granulate is stable and has an amorphous structure, such that an agglomeration into larger clusters or a growth into larger particles is prevented.

It has been found that it is particularly advantageous to convert the lipophilic component, or at least one of the lipophilic components, in particular the oil-/fat- and/or wax-containing components of the product into a granulate in accordance with the invention. In particular, the term “wax” includes animal and vegetable waxes as well as liquid waxes, such as jojoba oil. Other meltable components may certainly also be pulverized in accordance with the invention before being combined with the other components. Depending on the formulation in accordance with which the product is produced, it may even be advantageous to convert all components into a granulate before the mixing process.

However, it is often sufficient to bring the oil, fat and/or wax components into a granular state. After processing the granulated oil, fat and/or wax components with the other components, a product is obtained which is more stable than would be the case without the use according to the invention of granulated oils, fats or granulated waxes. During temperature fluctuations, the emulsions or suspensions produced do not decompose so quickly into their constituents or agglomerate, but instead maintain the mixed state.

The invention makes it possible to considerably reduce the amount of emulsifiers and stabilizers compared to a conventionally produced product. It may sometimes even be possible to completely omit emulsifiers and stabilizers. In this way the production costs can be reduced and a qualitative, high-grade product is also obtained which is more consumer-friendly. In particular consumers who are allergic to some additives will benefit from the products produced in accordance with the invention.

Once the granulate has been produced, the other components are advantageously processed further at a temperature of less than 25° C., preferably in a temperature range of 0° C. to 15° C. Compared to conventional methods, in which the components are often heated and then mixed together and processed at temperatures between 40° C. and 80° C., considerable energy savings can thus be made. Depending on which components are to be processed to form the product, temperatures of less than 0° C. may even be desirable.

The invention basically relates to the conversion of one or more liquid, pasty or solid components into a particulate, granular or pulverulent state. In this state the components can be better mixed with the other components and a much finer dispersion is obtained. Products which are particularly difficult to mix, such as water-oil components, can be much more easily converted into emulsions without the individual phases coalescing to form larger phase domains.

At least one component is preferably converted into a granulate which basically has particle sizes between 1 micrometer and 1000 micrometers, preferably between 1 micrometer and 300 micrometers. For this purpose the component is atomized or sprayed so finely in the liquid state of matter that correspondingly small droplets are formed which are then shock-cooled in accordance with the invention and are converted into the solid state of matter.

In a particularly preferred embodiment the fat components of a cosmetic product to be produced are brought into a fine amorphous granulate having a particle size of less than 300 micrometers. The use of this fine fat granulate facilitates the production of the desired emulsions or suspensions of which the product consists. The distribution of fat in the other components is much more homogeneous than in the usual production methods, in which the fat is mixed in the liquid state with the other components.

The invention has numerous advantages compared to the known production methods for cosmetic or pharmaceutical products. The fine granulate has very uniform particle sizes and is thus mixed very homogeneously with the other components so that the end product exhibits much greater stability. The granulate according to the invention is pourable, can be metered easily and can thus be processed further more easily, more quickly and more cost effectively. Owing to the fine dispersion of the individual components in the product, it is possible to reduce the amount of emulsifiers and stabilizers, and sometimes to even omit them completely. Products produced in this way exhibit greater quality. For example, care products produced in accordance with the invention are friendlier to skin and can also be produced more cost effectively.

The invention introduces advantages to the cosmetic industry in the case of production of all kinds of emulsions and suspensions. Some preferred fields of application are the production of
silane or silicone emulsions: these are used in cosmetics in the form of a gel or as an emulsion, in particular in skin creams and sun protection products;
gel: gels are dimensionally stable, easily deformable, finely dispersed systems formed of at least one solid and one aqueous phase. For example, gels are used as a carrier medium for ointments and creams or as hair gel;
oil-water emulsions, for example as used in sun protection sprays;
shampoo and hair care products;
creams and ointments containing a high fraction of fats and/or waxes.

The invention and further details thereof will be explained in greater detail hereinafter with reference to the embodiment illustrated in the drawing, in which:

the FIGURE shows a device for carrying out the method according to the invention.

The figure shows a device for producing fine oil or fat particles, which are then processed with further components to form cosmetics. The liquid fat or the oil is stored in a storage container 1. With the use of fat, this is heated beforehand until just above its melting point and is thus liquefied.

The oil or fat is conveyed via a line 3 to a two-component nozzle 4 by means of a pump 2 at a pressure between 1.5 and 8 bar. Gaseous nitrogen is also fed to the two-component nozzle 4 as atomizing gas via the line 5. The liquid fat or oil is mixed with the atomizing gas in the nozzle 4 and is emitted as a two-phase flow into a product container 11. The liquid fat or oil is present in this two-phase mixture as finely distributed droplets 6, preferably with a droplet size between 10 and 500 micrometers. The droplet size may be controlled by varying the pressure of the atomizing gas and/or the throughput of liquid fat or oil.

The fat or oil droplets are cooled by heat exchange with cryogenic nitrogen. For this purpose liquid nitrogen is fed via a line 7 to a closed-circuit pipeline 8. The closed-circuit pipeline 8 is arranged concentrically about the two-component nozzle 4 and has a number of discharge openings 9 in its underside, from which fine jets 10 of liquid nitrogen are ejected. The jets 10 of cryogenic nitrogen are oriented obliquely downwards and inwards and a heat exchange takes place between said jets and the fat or oil droplets 6, wherein the fat or oil droplets 6 harden to form small particles 12 and collect on the base of the product container 11. The liquid nitrogen used as a coolant evaporates and is suctioned off together with the atomizing gas via an offtake 13.

The solid fat or oil particles are then used as a starting material for the production of cosmetics.

1. A method for producing a cosmetic or pharmaceutical product, wherein a plurality of components are processed to form the product, comprising atomizing at least one component in a liquid state, contacting the at least one component in the liquid state by direct heat exchange with a cryogenic liquid gas to shock-cool the at least one component into a granulate, and processing the granulate with further components to form said product.

2. The method according to claim 1, wherein the atomizing produces a spray of fine droplets swirling through the cryogenic liquid gas.

3. The method according to claim 1, wherein the at least one component comprises a lipophilic substance selected from the group consisting of a fat and an oil converted into the granulate.

4. The method according to claims 1, wherein the processing of the granulate with the further components is at a temperature of less than 25° C., or in the temperature range from 0° C. to 15° C.

5. The method according to claim 4, wherein the processing of the granulate with the further components is at a temperature of less than 0° C.

6. The method according to claim 1, wherein the granulate comprises a particle size between 1 and 1000 micrometers.

7. The method according to claim 6, wherein the granulate comprises a particle size between 1 and 300 micrometers.

8. The method according to claim 1, wherein the processing of the granulate with the further components excludes emulsifiers or stabilizers.

9. The method according to claim 1, wherein said product produced comprises a cream or a paste.

10. The method according to claim 1, wherein the cryogenic liquid gas comprises liquid nitrogen.
